(19) United States
(12) Patent Application Publication
(10) Pub. No.: US 2025/0032294 A1
(43) Pub. Date: Jan. 30, 2025

(54) COMPRESSIVE ORTHOTIC DEVICE AND METHODS OF USE THEREOF

(71) Applicant: Quorum Orthopedics, Inc., Windsor, CO (US)

(72) Inventor: Joe Johnson, Windsor, CO (US)

(21) Appl. No.: 18/787,496

(22) Filed: Jul. 29, 2024

Related U.S. Application Data
(60) Provisional application No. 63/529,600, filed on Jul. 28, 2023.
(60) Provisional application No. 63/531,542, filed on Aug. 8, 2023.

(51) Int. Cl. A61F 5/01

(52) U.S. Cl. CPC A61F 5/0111; A61F 2005/0172

(57) ABSTRACT

A compressive orthotic device having a disarticulated compression insert having a collapsible scaffold matrix positioned below the calcaneus of a user.

### CROSS-REFERENCE TO RELATED APPLICATIONS

This U.S. Non-Provisional application claims the benefit of and priority to U.S. Provisional Application No. 63/529,600, filed Jul. 28, 2023, and U.S. Provisional Application No. 63/531,542, filed Aug. 8, 2023, the specification, claims and drawings of which are incorporated herein by reference in their entirety.

### TECHNICAL FIELD

Generally, the inventive technology disclosed herein relates to the field of orthotic devices, and in particular a compressive orthotic device having a disarticulated compression insert having a collapsible scaffold matrix positioned below the calcaneus of a user.

### BACKGROUND

Orthotics are generally considered to encompass any device that provides external biomechanical support to a human or animal body surface. Orthotics typically provide cushioning, arch support, and pronation control. A foot orthosis generally takes the form of an insole for a shoe. The insole has a region of support and/or relief. The region of support may be a profiled region designed to support the foot and provide correct positioning of the foot. The region of relief may be a soft/cushioned region aimed to reduce a force applied to a specific region of a patient's foot so as to relieve pain. A foot orthosis may be used to support a foot post-surgery, to improve the gait of a user, and/or to provide relief from conditions such as pressure ulcers.

There exists a variety of orthotic inserts, ranging from custom orthotic inserts prepared by medical practitioners to off-the-shelf varieties, such as foot pads, cushioning insoles and the like. Certain of these inserts may be geared more toward improving arch support, so that the arch undergoes fewer traumas or stress, especially during running or other physical activities. Other shoe inserts and orthotics may address long-term general comfort issues and focus on improving the cushioning of the associated shoe or athletic footwear, again, with the goal of reducing foot fatigue which may develop when the wearer is “on their feet” for extended periods of time. Still other inserts may focus on returning energy during running or walking, by providing a springiness or spring force, generally directed through the user's heel, with the thought that such energy return would improve speed or athletic performance.

Traditional orthotic devices often do not adequately factor in foot or heel anatomy or the associated dynamics and are not customizable with respect to the level of support and compression provided by the device. As such, there exists a long-felt need for an improved orthotic device having a customizable level of compression that can be adapted to a user's specification.

### SUMMARY OF THE INVENTION

In one aspect, the present invention includes a compression orthotic device having a compression insert configured to compresses in response to an externally applied force, such as a heel-strike accomplished while walking. In a preferred aspect, the orthotic device of the invention compression orthotic device of the invention includes an orthotic, and preferably a foot orthotic forming an orthotic body comprising a toe portion, a mid-foot portion and a heel portion. In this preferred aspect, the heel portion can include a heel aperture configured to secure a compression insert.

In a preferred aspect, the compression insert of the invention can include a disarticulated and/or integral compressive component having an external heel surface and a collapsible scaffold matrix configured to extend through the heel aperture and compresses in response to an externally applied force, such as a heel strike.

Further objects of the inventive technology will become apparent from the description and drawings below.

### BRIEF DESCRIPTION OF THE FIGURES

The inventive disarticulated compression socket described with reference to the accompanying drawings which show preferred embodiments according to the device described herein. It will be noted that the device as disclosed in the accompanying drawings is illustrated by way of example only. The various elements and combinations of elements described below and illustrated in the drawings can be arranged and organized differently to result in embodiments which are still within the spirit and scope of the device described herein.

FIG. 1A-B: (A) shows an isolated compression insert having a collapsible scaffold matrix in one embodiment thereof; (B) shows an isolated orthotic body having a heel, mid-foot and toe portions in one embodiment thereof;

FIG. 2: shows a top perspective view of a compression orthotic having a compression insert positioned within a heel aperture in one embodiment thereof;

FIG. 3: shows a side view of a compression orthotic having a compression insert positioned within a heel aperture such that a collapsible scaffold matrix extends below the heel portion of the orthotic body in one embodiment thereof;

FIG. 4: shows a rear view of a compression orthotic having a compression insert positioned within a heel aperture such that a collapsible scaffold matrix extends below the heel portion of the orthotic body in one embodiment thereof;

FIG. 5: shows a bottom view of a compression orthotic having a compression insert positioned within a heel aperture such that a collapsible scaffold matrix extends below the heel portion of the orthotic body in one embodiment thereof;

FIG. 6: shows an isolated compression insert having a collapsible scaffold matrix in one embodiment thereof;

FIG. 7: shows a top view of a compression orthotic having a disarticulate compression insert positioned adjacent to a heel aperture in one embodiment thereof;

FIG. 8: shows a bottom view of a pair of compression orthotics each having a compression insert positioned within a heel aperture such that a collapsible scaffold matrix extends below the heel portion of the orthotic body in one embodiment thereof;

FIG. 9: shows a top perspective view of a compression orthotic having a compression insert positioned within a heel aperture in one embodiment thereof;

FIG. 10: shows a top respective view of a compression orthotic having a compression insert secured by a pair of heel arm in one embodiment thereof;

FIG. 11: shows a bottom perspective view of a compression orthotic having a compression insert secured by a pair of heel arm with a collapsible scaffold matrix extending beyond the underside of the orthotic body in one embodiment thereof;

FIG. 12: shows a top perspective view of a compression orthotic having a disarticulated compression insert having a border containing a series of alternative slots and extensions positioned adjacent to a pair of heel arms having corresponding extension and slot positions in one embodiment thereof; and

FIG. 13: shows a side view of a compression orthotic having a compression insert secured by a pair of heel arm with a collapsible scaffold matrix extending beyond the underside of the orthotic body in one embodiment thereof.

### DETAILED DESCRIPTION OF THE INVENTION

The present invention includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

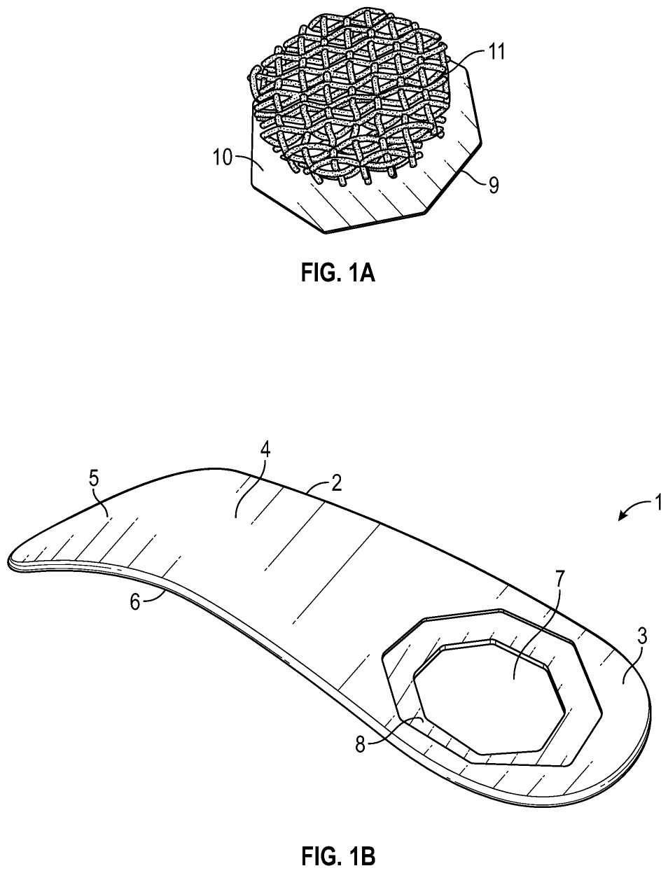

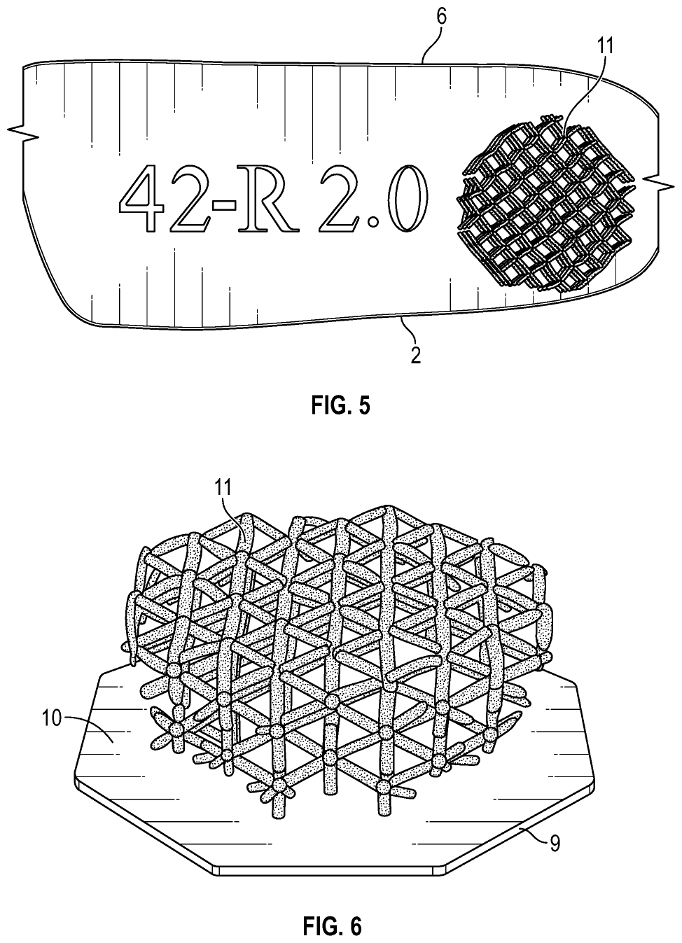

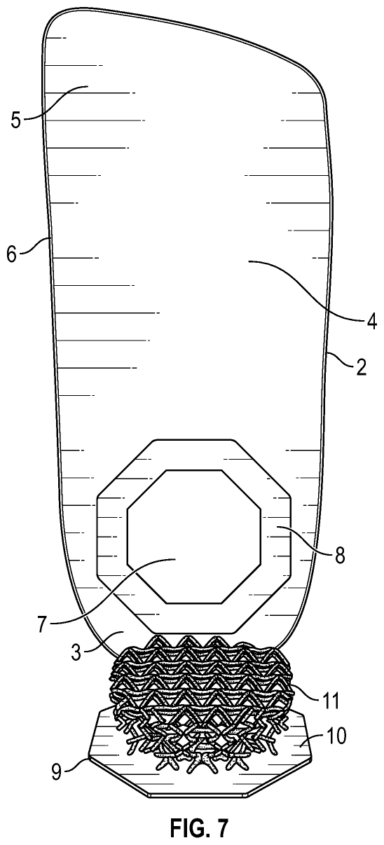

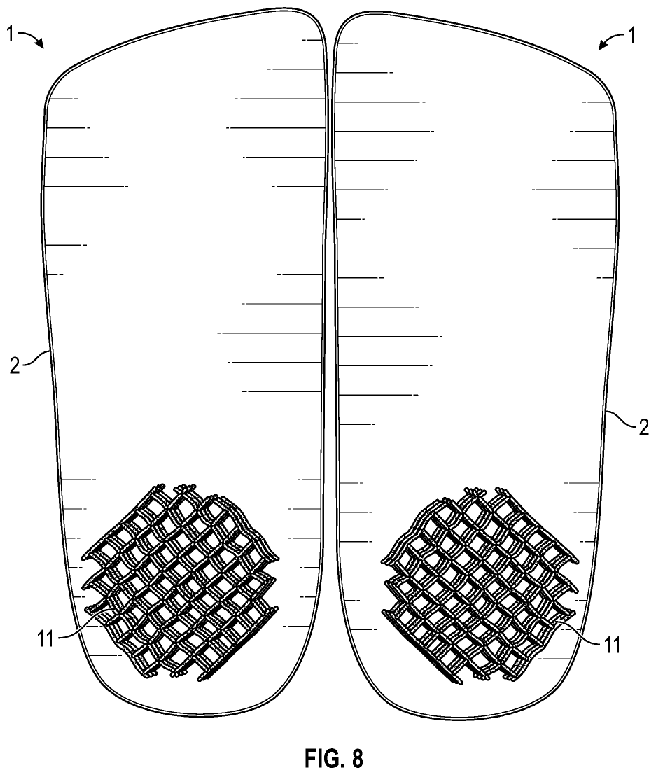

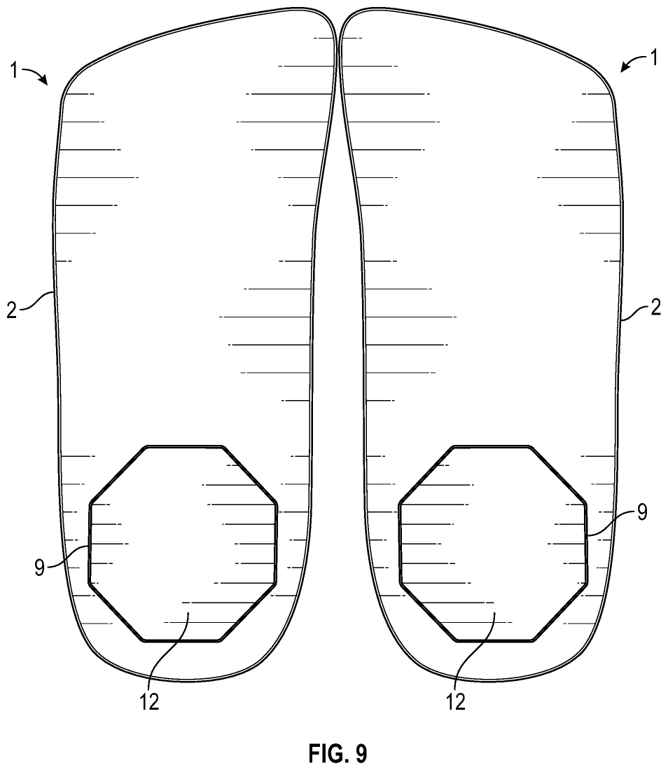

The present invention includes systems, methods, and devices for an improved compression orthotic (1). As shown in the preferred embodiment of FIGS. 1-9, the compression orthotic (1) of the invention includes a foot orthotic having that is configured to confirm to the posterior surface of a user's foot. The foot orthotic can be formed by an orthotic body (2) having a top (I) and bottom surface (II), as well as defined portions along the length of the orthotic, namely a heel portion (3), a mid-foot portion (4), and toe portion (5), and optionally an arch support (6) forming an orthotic body (2). The orthotic body (2) of the invention can be made from a variety of materials, such as plastics, various thermoplastics, rubber, gel, mesh, and silicone as well as various appropriate compressible materials known in the art. As noted above, the orthotic body (2) can be custom made for an individual user or can be constructed in a generic form for use by any consumer. In a preferred embodiment, the orthotic body (2) is 3D printed, and preferably 3D printed in a customized shape and configuration to confirm to the posterior portion of a user's foot surface.

Generally referring to FIG. 1, the compression orthotic (1) of the invention includes one or more heel aperture(s) (7) positioned at the heel portion (3) of the device. The heel aperture (7) of the invention can be configured to receive a disarticulated compression insert (9). As further shown in FIGS. 1-3, a compression insert (9) of the invention includes a heel surface (12) configured to be positioned below the heel or calcaneus of the user, and further includes a collapsible scaffold matrix (11) configured to compresses in response to an externally applied force.

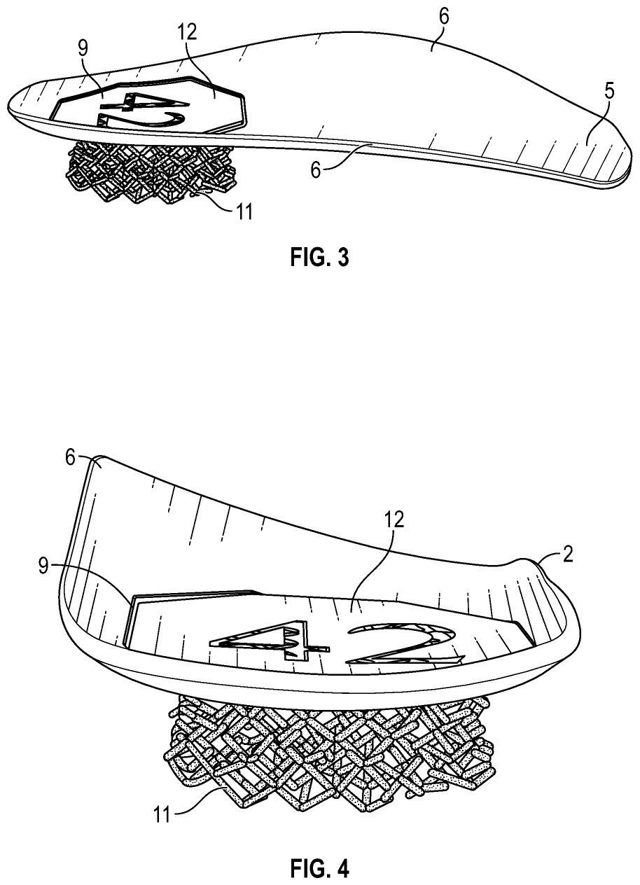

As shown in FIGS. 3-5, the compression insert (9) of the invention is secured to the heel portion (3) of the orthotic body (2) such that the collapsible scaffold matrix (11) extends through the heel aperture (7) and is in contact with the top surface of a user's shoe. In this embodiment, the collapsible scaffold matrix (11) is formed by a plurality of interconnected series of cantilevered supports that are configured to be compressed in response to an externally applied force. As noted below, the collapsible scaffold matrix (11) of the compression insert (9) may be formed of a material having sufficient elasticity/rigidity to allow it to be compressed in response to an external force and decompressed and return to its original configuration once that force has been removed, such a typical heel-strike that occurs while walking or running.

The application of a collapsible scaffold matrix (11) allows for the use of rigid materials for the contact surfaces, such as the heel surface, while maintaining sufficient elasticity in the collapsible scaffold matrix (11). Notably, a variety of matrix configurations or materials may be contemplated in this invention. For example, in certain alternative embodiments, the collapsible scaffold matrix (11) may include a solid compressible material, such as a gel or soft plastic or other elastic material. In additional embodiments, a collapsible scaffold matrix may include a series of springs or other supports that are positioned below the heel surface (12), and further configured to be compressed in response to an externally applied force, such as applied by contact of the heel with the collapsible scaffold matrix (11) during a heel strike, in one embodiment.

The compression insert (9), and collapsible scaffold matrix (11) can be an integral composition or may be further disarticulated. Moreover, the compression insert (9), and collapsible scaffold matrix (11) of the invention can be made from a variety of materials, such as plastics, various thermoplastics, rubber, gel, mesh, and silicone as well as various appropriate compressible materials known in the art. As noted above, the size, shape and compressibility of the compression insert (9), and in particular collapsible scaffold matrix (11) can be custom made for an individual user or can be constructed in a generic form for use by any consumer. In a preferred embodiment, the compression insert (9), and in particular collapsible scaffold matrix (11) is 3D printed, and preferably 3D printed in a customized shape and configuration to confirm to the posterior portion of a user's foot surface. In this embodiment, a user can select and use a customized compression insert (9) having a customized level of compression, for example based on the size, and configuration of the collapsible scaffold matrix (11) in one embodiment.

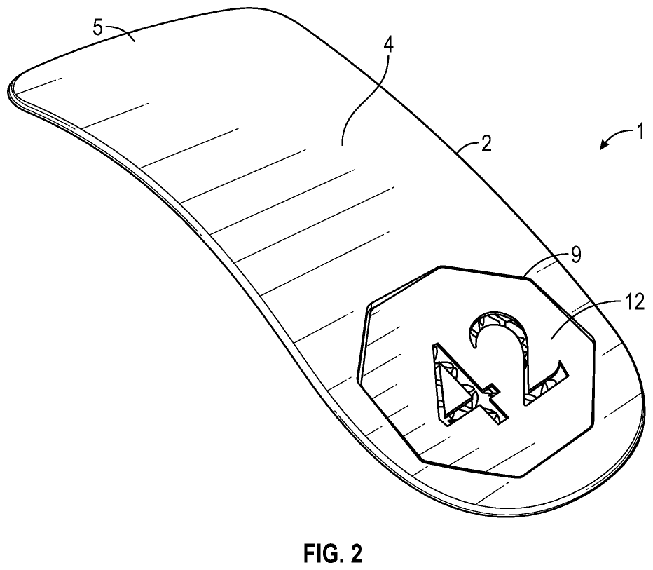

Referring again to FIG. 1, the heel aperture (7) of the invention can further include a channel (8) formed by a depression positioned around the heel aperture (7). As shown in FIG. 2, in a preferred embodiment, the compression insert (9) of the invention includes an extended channel surface (10) that can be positioned within the channel (8) such that the compression insert (9) is flush with the heel portion (3) of the orthotic body. In a certain embodiment, the channel surface (10) can be secured within the channel (8) with an adhesive.

In another embodiment, the compression orthotic device (2) can include an orthotic body (2) configured to secure a plurality of compression inserts (9) each having a collapsible scaffold matrix (11) configured to compresses in response to an externally applied force. In this embodiment, the compression inserts (9) can be secured to the orthotic body (2) at positions along the heel portion, mid-foot portion (4), toe portion (5) or arch support (6). As described above, in this embodiment, each compression insert (9) can be positioned in aperture such that the collapsible scaffold matrix (11) of each insert (9) extends outward from the bottom surface of the orthotic body (2).

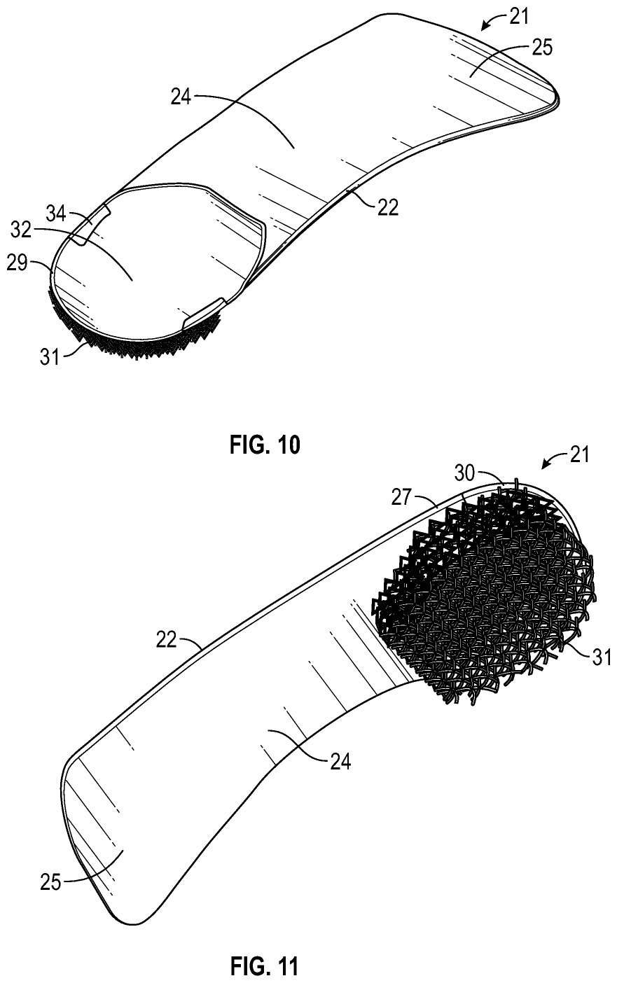

In an alternative embodiment, the present invention includes systems, methods, and devices for an improved compression orthotic (21). As shown in the preferred embodiment of FIGS. 10-13, the compression orthotic (21) of the invention includes a foot orthotic having that is configured to confirm to the posterior surface of a user's foot. The foot orthotic can be formed by an orthotic body (22) having a top (I) and bottom surface (II), as well as defined portions along the length of the orthotic, namely a heel portion (23) comprising a pair of heel arms (27) extending along the side edges of the heel portion (23) forming an open aperture positioned as described below, a mid-foot portion (24), and toe portion (25), and optionally an arch support (26) forming an orthotic body (22). The orthotic body (22) of the invention can be made from a variety of materials, such as plastics, various thermoplastics, rubber, gel, mesh, and silicone as well as various appropriate compressible materials known in the art. As noted above, the orthotic body (22) can be custom made for an individual user or can be constructed in a generic form for use by any consumer. In a preferred embodiment, the orthotic body (22) is 3D printed, and preferably 3D printed in a customized shape and configuration to confirm to the posterior portion of a user's foot surface.

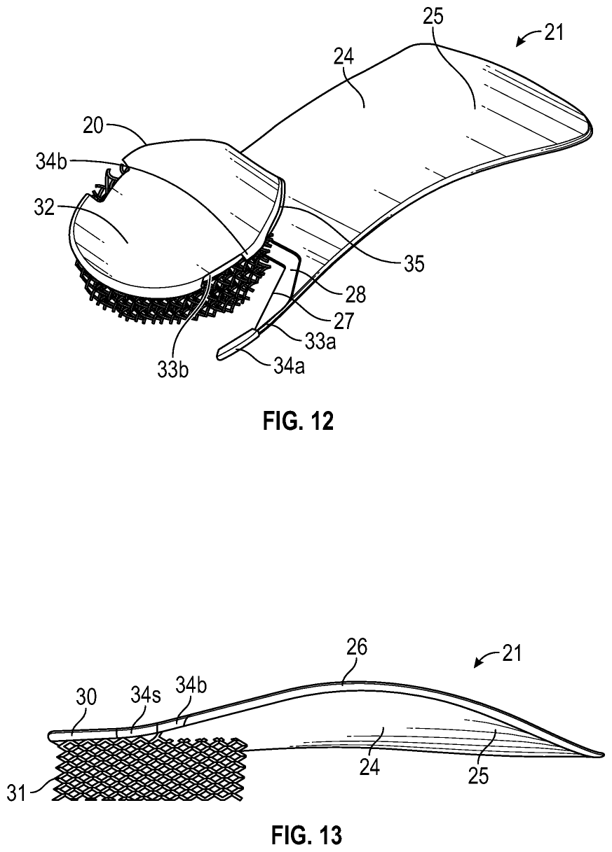

Generally referring to FIGS. 10-11, the compression orthotic (21) of the invention includes a pair of heel arms (27) positioned along the outside of the heel portion (23) forming an aperture configured to secure a compression insert (29) as described below. Again, as showing in FIGS. 12-13, in a preferred embodiment, a compression insert (29) includes a border (3) positioned long the outer heel surface (32) of the insert which is further configured to be secured to the heel arms (27) positioned along the outside of the heel portion (23). In this embodiment, the border (30) of the compression insert (29) can include one or preferably plurality of slots (33b) and extensions (34b) that correspond with opposing extensions (33a) and slots (33a) on each of the heel arms (27), respectively.

In this configuration, a compression insert (29) having a heel surface (32) is configured to confirm to the heel of a user's foot such that the collapsible scaffold matrix (31) compresses in response to an externally applied force. The compression insert (29) can be secured to the heel arms (27) such that the collapsible scaffold matrix (31) extends below the bottom surface of the orthotic body (22) forming a support structure for the user's foot while walking or running. In this preferred embodiment, the border (30) of the compression insert (30) includes one or more slots (33b) or extensions (34a) configured to be mated with a corresponding extension (34a) or slot (33a) on each of said heel arms (27). In an additional embodiment, the compression insert (29) of the invention includes a lip (35) extending outward from the compression insert (29) and further configured to be secured in a channel (28), for example by an adhesive or other coupler, such as a push or beveled coupler device, positioned on the front leading surface of the heel portion (23) or the orthotic body (22).

As further shown in FIGS. 10-13, the compression insert (29) of the invention includes a heel surface (32) configured to be positioned below the heel or calcaneus of the user, and further includes a collapsible scaffold matrix (31) configured to compresses in response to an externally applied force. In a preferred embodiment, the compression insert (29) of the invention is secured to the heel portion (23) of the orthotic body (22) such that the collapsible scaffold matrix (31) extends through the heel aperture formed by the heel arms (27) and is in contact with the top surface of a user's shoe. In this embodiment, the collapsible scaffold matrix (31) is formed by a plurality of interconnected series of cantilevered supports that are configured to be compressed in response to an externally applied force. As noted below, the collapsible scaffold matrix (31) of the compression insert (29) may be formed of a material having sufficient elasticity/rigidity to allow it to be compressed in response to an external force and decompressed and return to its original configuration once that force has been removed, such a typical heel-strike that occurs while walking or running.

The application of a collapsible scaffold matrix (31) allows for the use of rigid materials for the contact surfaces, such as the heel surface, while maintaining sufficient elasticity in the collapsible scaffold matrix (31). Notably, a variety of matrix configurations or materials may be contemplated in this invention. For example, in certain alternative embodiments, the collapsible scaffold matrix (31) may include a solid compressible material, such as a gel or soft plastic or other elastic material. In additional embodiments, a collapsible scaffold matrix may include a series of springs or other supports that are positioned below the heel surface (32), and further configured to be compressed in response to an externally applied force, such as applied by contact of the heel with the collapsible scaffold matrix (31) during a heel strike, in one embodiment.

The compression insert (29), and collapsible scaffold matrix (31) can be an integral composition or may be further disarticulated. Moreover, the compression insert (29), and collapsible scaffold matrix (31) of the invention can be made from a variety of materials, such as plastics, various thermoplastics, rubber, gel, mesh, and silicone as well as various appropriate compressible materials known in the art. As noted above, the size, shape and compressibility of the compression insert (29), and in particular collapsible scaffold matrix (31) can be custom made for an individual user or can be constructed in a generic form for use by any consumer. In a preferred embodiment, the compression insert (29), and in particular collapsible scaffold matrix (31) is 3D printed, and preferably 3D printed in a customized shape and configuration to confirm to the posterior portion of a user's foot surface. In this embodiment, a user can select and use a customized compression insert (29) having a customized level of compression, for example based on the size, and configuration of the collapsible scaffold matrix (11) in one embodiment.

Naturally, all embodiments discussed herein are merely illustrative and should not be construed to limit the scope of the inventive technology consistent with the broader inventive principles disclosed. As may be easily understood from the foregoing, the basic concepts of the present inventive technology may be embodied in a variety of ways. It generally involves systems, methods, techniques as well as devices to accomplish an improved compressive orthotic device. In this application, the methods and apparatus for the aforementioned systems are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the statements of invention. As can be easily understood from the foregoing, the basic concepts of the present invention may be embodied in a variety of ways. It involves both techniques as well as devices to accomplish the appropriate system. In this application, the techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention may be described in some instances in method-oriented terminology, each element of the claims corresponds to a device and vice versa. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing the explicit embodiment(s) shown, the great variety of implicit alternative embodiments, and the broad methods or processes and the like are encompassed by this disclosure and may be relied upon when drafting any claims. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “support” should be understood to encompass disclosure of the act of “supporting”—whether explicitly discussed or not—and, conversely, were there effectively disclosure of the act of “supporting”, such a disclosure should be understood to encompass disclosure of a “supporting method and/or technique, and/or device” and even a “means for supporting.” Such changes and alternative terms are to be understood to be explicitly included in the description.

Any patents, publications, or other references mentioned in this application for patent, such as in the specification or an IDS are hereby incorporated herein by reference in their entirety. Any priority case(s) claimed by this application is hereby appended and hereby incorporated herein by reference in their entirety. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated herein by reference in their entirety. Finally, all references listed in the list of References To Be Incorporated By Reference In Accordance With The patent application or other information disclosure statement and the like filed with the application are hereby appended and hereby incorporated herein by reference in their entirety, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the methods and/or apparatus for providing a compression orthotic device as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, xii) each potentially dependent claim or concept as a dependency on each and every one of the independent claims or concepts presented, and xiii) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this, or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. It should be understood that this application also provides support for any combination of elements in the claims and even incorporates any desired proper antecedent basis for certain claim combinations such as with combinations of method, apparatus, process, and the like claims.

Any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The inventive subject matter is to include, but certainly not be limited as, a system substantially as herein described with reference to any one or more of the Figures and Description (including the following: for example, the process according to any claims and further comprising any of the steps as shown in any Figures, separately, in any combination or permutation).

Finally, Applicant reserves the right to seek additional design patent protections over the claimed invention; such that the drawings are fully enabled so as to allow one of ordinary skill in the art to know that the claimed design was in Applicant's possession at the time of filing. As such, it should be noted that any broken lines are to be included for the purpose of illustrating environmental matter and form no part of the claimed design should such become necessary.

What is claimed is:

1. A compression orthotic device comprising:
an orthotic body having a toe portion, a mid-foot portion and a heel portion;
a heel aperture positioned at the heel portion;
a compression insert having a heel surface and collapsible scaffold matrix configured to compresses in response to an externally applied force, wherein the insert is secured to the heel portion of the orthotic body such that the collapsible scaffold matrix extends through the heel aperture.

2. The device of claim 1, wherein said heel surface is secured to a channel positioned adjacent to the heel aperture.

3. The device of claim 1, wherein said heel surface is secured to a channel positioned adjacent to the heel aperture by an adhesive.

4. The device of claim 1, wherein said heel surface is positioned below the calcaneus of a user.

5. The device of claim 1, wherein said collapsible scaffold matrix comprises a plurality of interconnected series of cantilevered supports that are configured to be compressed in response to an externally applied force.

6. The device of claim 1, wherein said orthotic body and said compression insert comprise an integral compression orthotic device.

7. The device of claim 6, wherein said integral compression orthotic device comprises a 3D printed integral compression orthotic device.

8. A compression orthotic device comprising:
an orthotic body having a top and bottom surface; and
a plurality of compression inserts each having a collapsible scaffold matrix configured to compresses in response to an externally applied force, wherein the insert is secured to the orthotic body such that the collapsible scaffold matrix extends outward from the bottom surface of the orthotic body.

9. The device of claim 8, wherein at least one of said plurality of compression inserts is secured to a channel positioned adjacent to a heel aperture.

10. The device of claim 8, wherein said collapsible scaffold matrix on said of plurality compression inserts comprises a plurality of interconnected series of cantilevered supports that are configured to be compressed in response to an externally applied force.

11. The device of claim 8, wherein said orthotic body and said the plurality compression inserts comprise an integral compression orthotic device.

12. A compression orthotic device comprising:
an orthotic body having a toe portion, a mid-foot portion and a heel portion, the heel portion comprising a pair of heel arms;
a compression insert having a heel surface and collapsible scaffold matrix configured to compresses in response to an externally applied force, wherein the insert is secured to the heel arms such that the collapsible scaffold matrix extends below the bottom surface of the orthotic body.

13. The device of claim 12, wherein said compression insert comprises a border having one or more slots or extensions configured to be mated with corresponding slots or extension on each of said heel arms.

14. The device of claim 12, wherein said heel portion comprises a channel.

15. The device of claim 14, wherein said compression insert comprises an extended lip configured to be secured in said channel.

16. The device of claim 14, wherein said compression insert comprises an extended lip configured to be secured in said channel by an adhesive.

17. The device of claim 12, wherein said heel surface is positioned below the calcaneus of a user.

18. The device of claim 12, wherein said collapsible scaffold matrix comprises a plurality of interconnected series of cantilevered supports that are configured to be compressed in response to an externally applied force.

19. The device of claim 12, wherein said orthotic body and said compression insert comprise an integral compression orthotic device.

20. The device of claim 12, wherein said integral compression orthotic device comprises a 3D printed integral compression orthotic device.
